(19) United States
(12) Patent Application Publication
(10) Pub. No.: US 2003/0217943 A1
(43) Pub. Date: Nov. 27, 2003

(54) Dual chamber specimen bag

(76) Inventor: Neil Sklar, East Rockaway, NY (US)

Correspondence Address: David M. Carter, Carter, DeLuca, Farrell & Schmidt, LLP, Suite 225, 445 Broad Hollow Road, Melville, NY, 11747, US

(21) Appl. No.: 10/444,034

(22) Filed: May 22, 2003

Related U.S. Application Data
(60) Provisional application No. 60/382,456, filed on May 22, 2002.

(51) Int. Cl. A61L 15/00

(52) U.S. Cl. 206/438; 383/93

(57) ABSTRACT

The present disclosure relates to medical specimen bags. In one aspect of the present disclosure, a specimen bag is provided and includes a front panel having an upper terminal edge, an intermediate panel having an upper terminal edge which extends beyond the upper terminal edge of the front panel, the front panel and the intermediate panel sharing a common lower terminal edge, the front panel and the intermediate panel defining a first chamber, and a rear panel having an upper terminal edge and a lower terminal edge, the upper terminal edge of the rear panel extending beyond the upper terminal edge of the front panel and the lower terminal edge of the rear panel terminating before the lower terminal edge of the front and intermediate panels, the rear panel and the intermediate panel defining a second chamber.

### CROSS-REFERENCE TO RELATED APPLICATIONS

The present application claims the benefit of and priority to U.S. Provisional Patent Application Serial No. 60/382,456 filed on May 22, 2002, the entire content of which is incorporated herein by reference.

### BACKGROUND

1. Technical Field

The present disclosure relates to medical specimen bags and, more particularly, to a closeable plastic film bag having a first sealable chamber for receiving and retaining an item and a second scalable chamber for receiving and retaining documents relating to the item retained with the first chamber.

2. Background of Related Art

Plastic film bags are manufactured and are very commonly used for holding and storing various types of items ranging from industrial and manufacturing items to edible goods and medical specimens. Such bags are generally made by joining thin plastic film panels so as to form the bag and range in size and holding capacity. One type of plastic film bag which is today commonly used is initially open for placing a product or item therein and is, thereafter, easily selectively closeable. It is desirable that the closure seal be generally secure and, after a product has been placed therein and the bag closed, when desired, the bag can be opened, generally by cutting or tearing at serration or score lines, for gaining access to the product therein.

In general, the typical plastic film bag includes a front and a rear plastic wall joined together so as to form a bag having a bottom, side edges and an opening between the wall top edges. An adhesive strip is provided on the inside surface of one of the walls and extends linearly near the top edges from side edge to side edge. A release liner is provided extending the full width of the bag from side edge to side edge but is partially notched at its longitudinal ends for creating a shortened longitudinal portion which is placed within the bag between the front and rear walls and over the adhesive strip. Typically, the surface of the release linear which is to contact the adhesive strip is coated with a layer of silicone in order to prevent the release liner from sticking to the adhesive strip. By pulling the release liner out from between the front and rear walls, the adhesive strip is exposed to the inside surface of the other wall for thereby adhering or attaching together the front and rear walls along the bag opening and for closing the bag. The adhesive strip is made of an adhesive which is selected to be sufficiently tacky in order to prevent easy separation of the front wall from the rear wall and in order to prevent loss of the contents of the bag retained therein.

As is typically the case with such bags, once the front and rear walls have been adhered to one another a cutting implement is required in order to open the bag and gain access to the contents retained therein. Accordingly, the need exists for a plastic film bag which is liquid tight and which can be easily opened in order to gain access to the contents of the bag retained therein.

### SUMMARY

The present disclosure relates to medical specimen bags. In one aspect of the present disclosure, a specimen bag is provided and includes a front panel having an upper terminal edge, an intermediate panel having an upper terminal edge which extends beyond the upper terminal edge of the front panel, the front panel and the intermediate panel sharing a common lower terminal edge, the front panel and the intermediate panel defining a first chamber, and a rear panel having an upper terminal edge and a lower terminal edge, the upper terminal edge of the rear panel extending beyond the upper terminal edge of the front panel and the lower terminal edge of the rear panel terminating before the lower terminal edge of the front and intermediate panels, the rear panel and the intermediate panel defining a second chamber.

The specimen bag can further include a weld line extending along each of a right and a left side of the specimen bag. Preferably, from the upper terminal edge of the rear panel and the intermediate panel to the lower terminal edge of the front panel and the intermediate panels.

The specimen bag further includes a first adhesive seal disposed between the front and the intermediate panels for securely sealing the front panel against the intermediate panel. The specimen bag can further include a second adhesive seal disposed between the rear panel and intermediate panels for securely sealing the rear panel against the intermediate panel. The first and second adhesive seals extend between the right and left side weld lines.

The specimen bag further includes a release liner extending across and over each of the first and second adhesive seals, wherein the release liners prevent the first and second adhesive seals from prematurely closing the first and second chambers.

The specimen bag desirably includes a first set of score lines formed in and extending at least between the weld lines of the front panel and the intermediate panel. The first set of score lines is formed between the lower terminal edges of the front and intermediate panels and the lower terminal edge of the rear panel. The first set of score lines are preferably in apposition to one another.

The specimen bag can further include a second set of score lines formed in and extending at least between the weld lines of the rear panel and the intermediate panel. The second set of score lines is preferably formed between the first and the second adhesive seals.

It is envisioned that at least one of the first and second set of score lines is in the form of a grove defined by having a reduced thickness as compared to a thickness of the respective front and intermediate panel.

The first adhesive seal is applied to a surface of the intermediate panel in opposition to the front panel and at a location below the upper terminal edge of the front panel. Likewise, the second adhesive seal is applied to a surface of the intermediate panel in opposition to the rear panel and at a location below the upper terminal edges of the rear and intermediate panels.

The first and second adhesive seals are fabricated from a layer of pressure sensitive adhesive sealant material. Each release liner preferably has a length which is less than the length of the adhesive seal which it covers.

The presently disclosed specimen bag, together with attendant advantages, will be best understood by reference to the following detailed description in conjunction with the figures below.

### BRIEF DESCRIPTION OF THE DRAWINGS

The accompanying drawings, which are incorporated in and constitute a part of this specification, illustrate embodiments of the disclosure and, together with the general description given above, and the detailed description of the embodiments given below, serve to explain the principles of the present disclosure.

FIG. 1 is a perspective view of a dual chamber plastic film bag in accordance with the present disclosure;

FIG. 2 is a front elevational view of the dual chamber plastic film bag of FIG. 1;

FIG. 3 is a cross-sectional side elevational view of the dual chamber plastic film bag of the subject disclosure taken along line 3-3 of FIG. 2;

FIG. 4 is an enlarged localized view of the upper region of the dual chamber plastic film bag of FIG. 3 with a first release liner covering a first adhesive seal on an intermediate panel in order to prevent contact between the first adhesive seal and a front panel of the bag and a second release liner covering a second adhesive seal on a rear panel in order to prevent contact between the second adhesive seal and the intermediate panel;

FIG. 5 is an enlarged localized view of the lower region of the dual chamber plastic film bag of FIG. 3;

FIG. 6 is a localized view as in FIG. 4 with the first release liner removed from the bag to uncover the first adhesive seal in order to adhere the front panel of the bag to the intermediate panel to thereby enclose an article within a first chamber of the bag;

FIG. 7 is a localized view as in FIG. 4 with the second release liner removed from the bag to uncover the second adhesive seal in order to adhere the rear panel of the bag to the intermediate panel to thereby enclose an article a second chamber of the bag;

FIG. 8 is a perspective view of the dual chamber plastic film bag of FIG. 1 with the first chamber being at least partially open, in accordance with the subject disclosure, in order to remove an article therefrom; and

FIG. 9 is a perspective view of the dual chamber plastic film bag of FIG. 2 with the second chamber being at least partially open, in accordance with the subject disclosure, in order to remove an article therefrom.

### DETAILED DESCRIPTION OF PREFERRED EMBODIMENTS

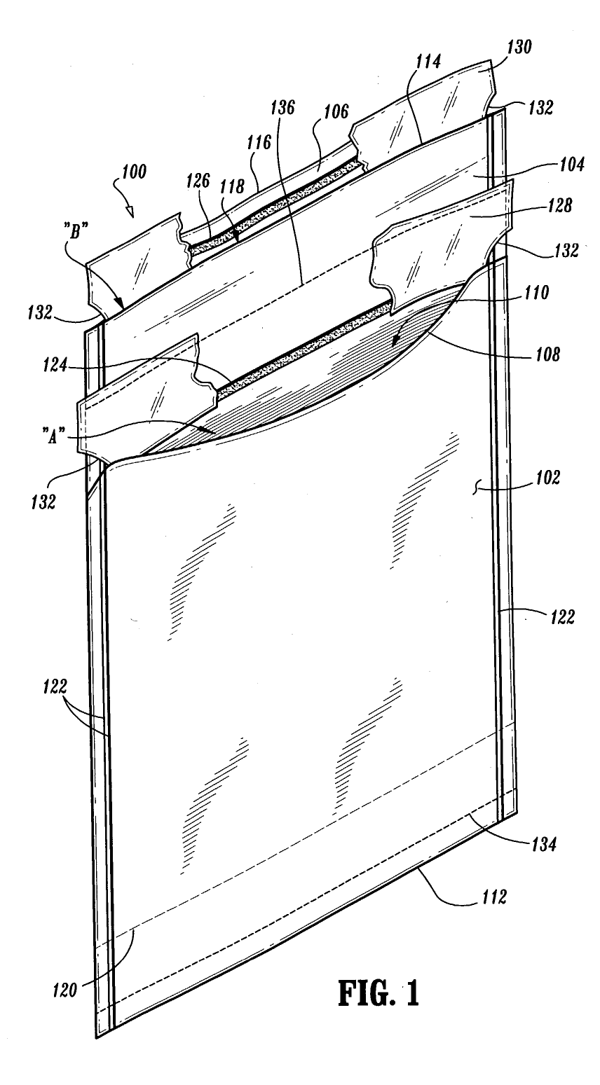

Preferred embodiments of the presently disclosed dual chamber plastic film bag will now be described in detail with reference to the drawing figures wherein like reference numerals identify similar or identical elements. Referring to FIGS. 1-9, a dual chamber plastic film bag is shown generally as reference numeral 100. Although bag 100 offers significant advantages and will be described in detail for use with medical specimens and samples, it will be understood that the bag is applicable for use in, for example, transporting money, checks, bonds, stocks, food stamps, jewelry and other such items.

In accordance with the present disclosure, bag 100 is formed from a medium-weight durable plastic such as polyethylene, polypropylene, polyethylene terephthalate, polyvinylchloride, vinyl acetate or other similar plastic material. A material thickness of about 0.002 mil or heavier is typical for such bags. It is envisioned that the bag material may be a single layer or multiple layers, and may be opaque, translucent or transparent.

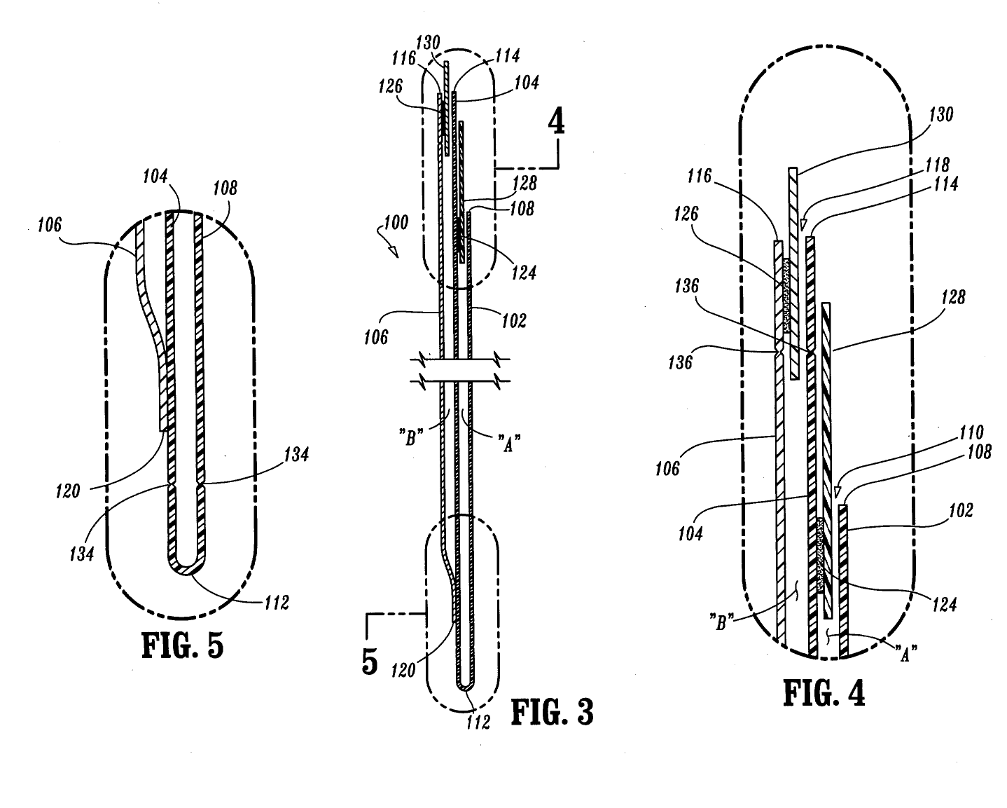

Referring now to FIGS. 1-4, bag 100 includes a front panel 102, an intermediate panel 104 and a rear panel 106. Front panel 102 includes an upper terminal edge 108 which together with intermediate panel 104 define an opening 110 for gaining access into a first chamber “A” of bag 100 (see FIG. 4). Preferably, front panel 102 and intermediate panel 104 are formed from a single piece of material folded over onto its self to define a common lower terminal edge 112. Intermediate panel 104 and rear panel 106 include an upper terminal edge 114 and 116, respectively, which extend beyond upper terminal edge 108 of front panel 102. Upper terminal edges 114 and 116 define an opening 118 for gaining access into a second chamber “B” of bag 100 (see FIG. 4). Rear panel 106 includes a lower terminal edge 120 spaced a distance from lower terminal edge 112 of front panel 102 and rear panel 104 (see FIGS. 3 and 5).

It is envisioned that chamber “A” is configured and adapted to receive and retain a specimen (i.e., a vial of blood, a vial of urine, a body tissue sample, or other medical sample). It is further envisioned that chamber “B” is configured and adapted to receive and retain paperwork (i.e., tags, identification cards/papers, instructional documents or the like) relating to the specimen contained in chamber “A”.

Preferably, bag 100 is formed by aligning upper terminal edge 114 of intermediate panel 104 and upper terminal edge 116 of rear panel 106 and either heat welding or fusing lower terminal edge 120 of rear panel 106 to intermediate panel 104. Front panel 102 is then folded along lower terminal edge 112 in a direction opposite the side of rear panel 106. The respective sides of front panel 102, intermediate panel 104 and rear panel 106 are then heat welded or fused together to define weld lines 122. Preferably, weld lines 122 extend from lower terminal edge 112 of front panel 102 and intermediate panel 104 through to upper terminal edges 114 and 116 of intermediate panel 104 and rear panel 106, respectively. In an alternative embodiment, it is contemplated that front panel 102 and intermediate panel 104 are separate discrete elements which are joined to one another along the respective bottom edges thereof by heat welding or fusing methods which are well known in the art.

Preferably, in accordance with the present disclosure, weld lines 122 are formed in such a manner so as to create a fluid tight seal running the entire length thereof. In this manner, fluid (i.e., air, gases, water, blood, urine, liquids, etc.) is prevented from entering or escaping across weld line 122.

With continuing reference to FIG. 1, bag 100 includes a pair of adhesive seals 124 and 126 for respectively securely sealing openings 110 and 118. First adhesive seal 124 includes a layer of pressure sensitive adhesive sealant material which is applied to the surface of intermediate panel 104, between front panel 102 and intermediate panel 104, at a location below upper terminal edge 108 of front panel 104. Preferably, first adhesive seal 124 is positioned to sealingly adhere to the interior surface of front panel 102. First adhesive seal 124 extends across the entire width of intermediate panel 104 though weld lines 122. It is envisioned that adhesive seal 124 can be applied to an inner surface of front panel 102 at a location just below terminal edge 108.

Second adhesive seal 126 includes a layer of pressure sensitive adhesive sealant material which is applied to the inner surface of rear panel 106 in close proximity to terminal edges 114 and 116. Second adhesive seal 126 extends across the entire width of rear panel 106 through wells lines 122. It is further envisioned that adhesive seal 126 can be applied to a surface of rear panel 106, between rear panel 106 and intermediate panel 104, at a location below terminal edges 114, 116, preferably between terminal edges 114, 116 and score lines 136 (see FIG. 4).

Bag 100 further includes a pair of release liners 128 and 130 which are formed of a strip of low adhesion plastic material suitable for use with the particular adhesive materials employed on the envelope. It is contemplated that each release liner 128, 130 be coated with a film of silicone or other non-stick substance in order to reduce the adhesive characteristics of release liner 128, 130. Release liners 128, 130 are configured and dimensioned to substantially cover adhesive seals 124 and 126, respectively, in their entireties to prevent adhesive seals 124, 126 from contacting the inner surface of front panel 102 and the rear surface of intermediate panel 106, respectively, before chambers “A” and “B” are closed, i.e., during packing, storing and handling.

In accordance with the present disclosure, each release liner 128, 130 includes a chamfer 132 formed at either end thereof. Stated differently, each release liner 128, 130 has a rear edge having a length equal to or greater than a width of panels 102, 104 and 106, and a front edge having a length less than the width of panels 102, 104 and 106. Preferably, release liners 128, 130 are centrally positioned over adhesive seals 124 and 126, respectively, such that terminal ends (i.e., ends located in proximity to weld lines 122) of adhesive seals 124, 126 are exposed. As such, front panel 102 is pre-adhered to intermediate panel 104 near weld lines 122 and rear panel 106 is also pre-adhered to intermediate panel 104 near weld lines 122.

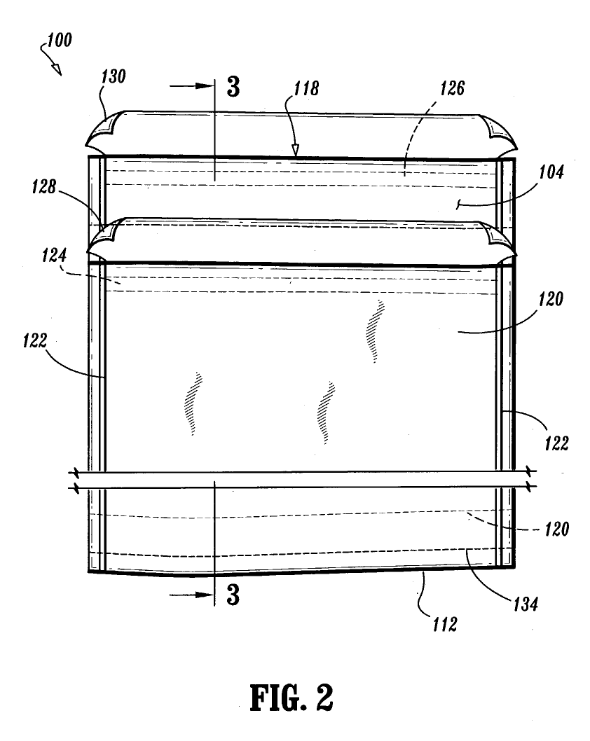

As seen in FIGS. 1 and 2, and in greater detail in FIG. 5, in accordance with the present disclosure, bag 100 further includes a first pair of score lines 134 formed near lower terminal edge 112 of front panel 102 and intermediate panel 104, wherein a first of the pair of score lines 134 is formed in front panel 102 and a second of the pair of score lines 134 is formed in intermediate panel 104. Preferably, the pair of score lines 134 are formed at a location between lower terminal edge 112 of front panel 102 and intermediate panel 104 and lower terminal edge 120 of rear panel 106. More preferably, the pair of score lines 134 are formed in apposition to one another.

Preferably, the pair of score lines 134 extend across the entire width of bag 100 and create a line of reduced strength in front panel 102 and intermediate panel 104. Score lines 134 are lines in which the thickness of the panels has been reduced (see FIG. 5) to be less than the preferred material thickness of the material making up bag 100 of about 0.002 mil. In accordance with the present disclosure and as seen in FIG. 5, it is preferred that front panel 102 and intermediate panel 104 be reduced in thickness on either side thereof (i.e., in the form of a double groove), however, it is contemplated that front panel 102 and intermediate panel 104 can be reduced in thickness along a single side thereof (i.e., in the form of a single groove).

As seen in FIGS. 1 and 2, and in greater detail in FIG. 4, in accordance with the present disclosure, bag 100 further includes a second pair of score lines 136, wherein a first of the pair of score lines 136 is formed near upper terminal edge 114 of intermediate panel 104 and a second of the pair of score lines 136 is formed near upper terminal edge 116 of rear panel 106. Preferably, the pair of score lines 136 are formed at a location between first adhesive seal 124 and second adhesive seal 126. More preferably, the pair of score lines 136 are formed in apposition to one another.

Preferably, the pair of score lines 136 extend across the entire width of bag 100 and create a line of reduced strength in intermediate panel 104 and rear panel 106. Score lines 136 are lines in which the thickness of the panels has been reduced (see FIG. 4) to be less than the preferred material thickness of the material making up bag 100 of about 0.002 mil. In accordance with the present disclosure and as seen in FIG. 4, it is preferred that intermediate panel 104 and rear panel 106 be reduced in thickness on either side thereof (i.e., in the form of a double groove), however, it is contemplated that intermediate panel 104 and rear panel 106 can be reduced in thickness along a single side thereof (i.e., in the form of a single groove).

In accordance with the present disclosure, score lines 134, 136 do not penetrate through panels 102, 104 and 106, thereby maintaining the integrity of bag 100. Unlike perforations, since score lines 134, 136 are a groove formed in panels 102, 104 and 106, fluid is prevented from entering into or escaping out of chambers “A” and/or “B”. Score lines 134 and 136 permit a user to easily open and gain access into chamber “A” and chamber “B”, respectively, by tearing along score lines 134 or 136.

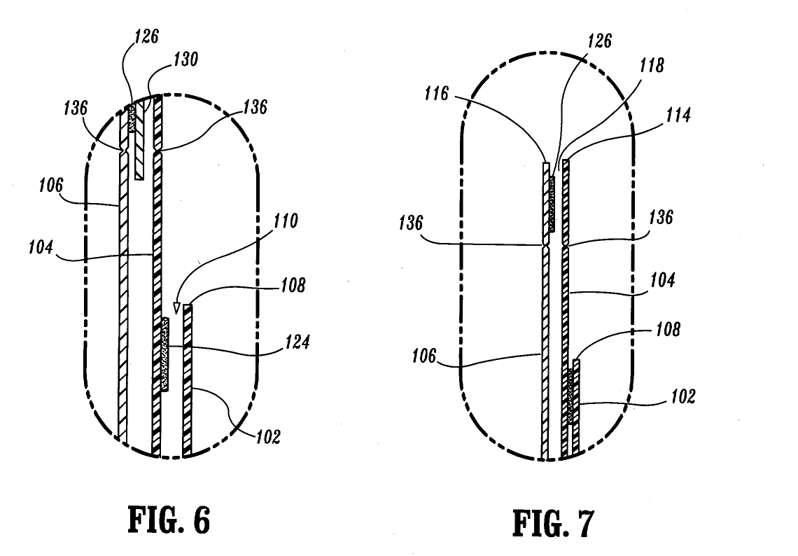

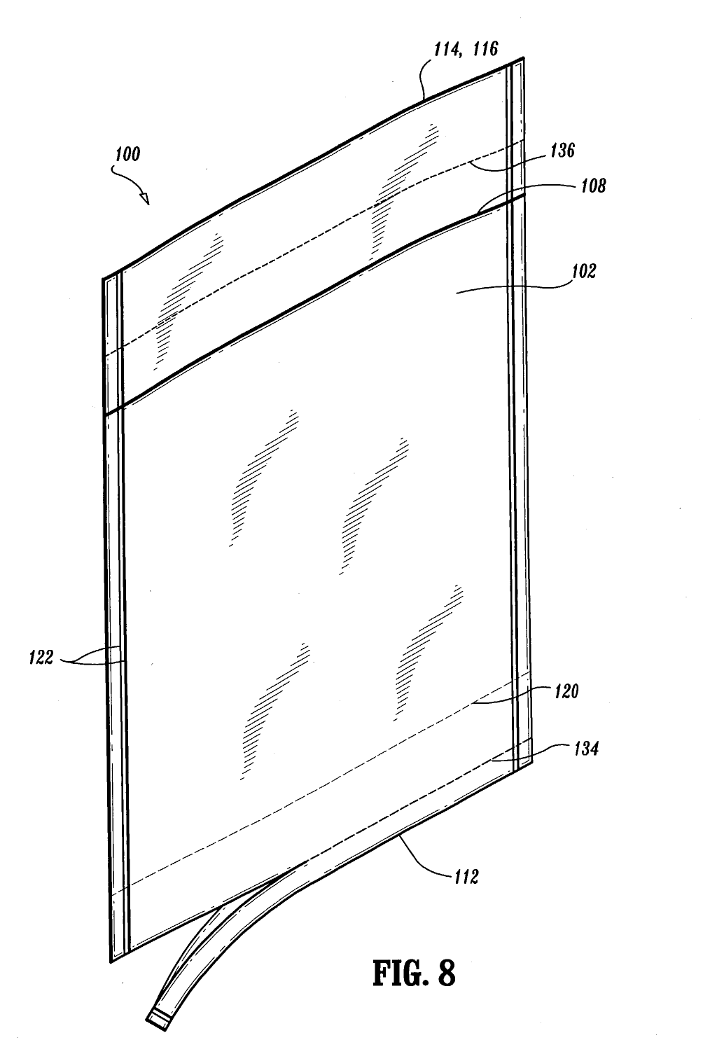

Referring now to FIGS. 1-9, use of bag 100 will now be discussed and described. To securely seal chamber “A” of bag 100, release liner 128 is removed from the protective position, i.e., a position overlying adhesive seal 124. At such a time, adhesive seal 124 is exposed and freed to contact the interior surface of front panel 102, as shown in FIG. 6. Subsequently, pressure is applied to front panel 102, along adhesive seal 124, in order to adhere front panel 102 to intermediate panel 104, as shown in FIG. 7. Chamber “A” is fluid tight since adhesive seal 124 extends completely across the width of bag 100, weld lines 122 extend from adhesive seal 124 through the bottom of bag 100, lower terminal edge 112 is preferably formed by folding front panel 102 over intermediate panel 104 and score lines 134 do not penetrate through front panel 102 and intermediate panel 104. Further, the adhesive making up adhesive seal 124 is such that the force required to gain access to chamber “A” along adhesive seal 124 is greater than the force required to gain access to chamber “A” along score lines 134. Thus, in order to most easily gain access to chamber “A”, as seen in FIG. 8, the user can tear bag 100 along score lines 134.

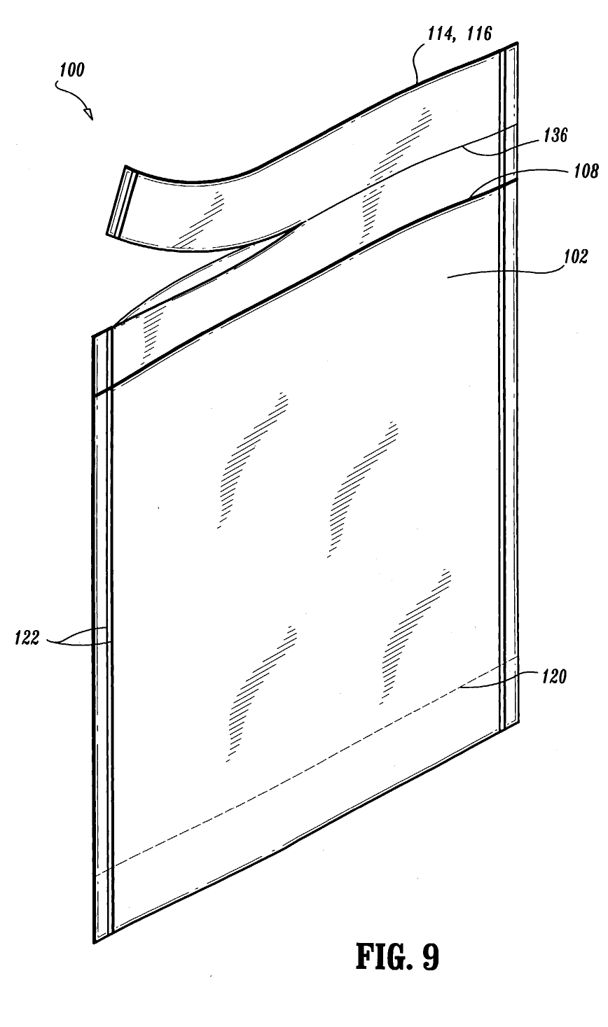

Similarly, to securely seal chamber “B” of bag 100, release liner 130 is removed from the protective position, i.e., a position overlying adhesive seal 126. At such a time, adhesive seal 126 is exposed and freed to contact the rear surface of intermediate panel 104, as shown in FIG. 7. Subsequently, pressure is applied to intermediate panel 104, along adhesive seal 126 in order to adhere intermediate panel 104 to rear panel 106. Chamber “B” is fluid tight since adhesive 126 extends completely across the width of bag 100, weld lines 122 extend from adhesive seal 126 through the bottom of bag 100, lower terminal edge 120 of rear panel 106 is welded or fused to intermediate panel 104 and score lines 136 do not penetrate through intermediate panel 104 and rear panel 106. Further, the adhesive making up adhesive seal 126 is such that the force required to gain access to chamber “B” along adhesive seal 126 is greater than the force required to gain access to chamber “B” along score lines 136. Thus, in order to most easily gain access to chamber “B”, as seen in FIG. 9, the user can tear bag 100 along score lines 136.

It is envisioned that bag 100 can be provided with tamper-evident systems that provide visually observable evidence of tampering. In particular, it is envisioned that the border regions of bag 100 are imprinted with geometric patterns that become distorted if attempts are made to open bag 100. Any distortion of the geometric patterns will indicate to the observer that bag 100 has been tampered with, and that its contents may have been compromised.

It is further envisioned that bag 100 can be provided with another tamper evident security mechanism which includes patterns of low adhesion indicia imprinted on the surface opposite adhesive seals 124, 126. The indicia is adapted to adhere to seals 124, 126 when chambers “A” and “B” of bag 100 are closed and seals 124, 126 contact their corresponding opposite surfaces. In use, when chambers “A” and “B” are sealed, the indicia adheres to seals 124, 126. Subsequently, when chambers “A” and/or “B” are opened along adhesive seals 124, 126, the indicia is visibly apparent on the surface of adhesive seals 124, 126, indicating to an observer that tampering has occurred.

It is further contemplated that each chamber “A” and “B” can be optionally provided with an insert of fluid absorbent or odor absorbent material disposed therein. Accordingly, in the event that a specimen or a vial containing a fluid is broken within either of chambers “A” or “B”, absorbent insert will act like a sponge to absorb the fluid and prevent the fluid from spreading once chamber “A” or “B” has been opened.

Although the subject disclosure has been described with respect to preferred embodiments, it will be readily apparent to those having ordinary skill in the art to which it appertains that changes and modifications may be made thereto without departing from the spirit or scope of the subject disclosure.

What is claimed is:

1. A specimen bag, comprising:
a front panel having an upper terminal edge;
an intermediate panel having an upper terminal edge which extends beyond the upper terminal edge of the front panel, the front panel and the intermediate panel sharing a common lower terminal edge, the front panel and the intermediate panel defining a first chamber; and
a rear panel having an upper terminal edge and a lower terminal edge, the upper terminal edge of the rear panel extending beyond the upper terminal edge of the front panel and the lower terminal edge of the rear panel terminating before the lower terminal edge of the front and intermediate panels, the rear panel and the intermediate panel defining a second chamber.

2. The specimen bag according to claim 1, further including a weld line extending along each of a right and a left side of the specimen bag, from the upper terminal edge of the rear panel and the intermediate panel to the lower terminal edge of the front panel and the intermediate panels.

3 The specimen bag according to claim 2, further comprising a first adhesive seal disposed between the front and the intermediate panels for securely sealing the front panel against the intermediate panel.

4. The specimen bag according to claim 3, further comprising a second adhesive seal disposed between the rear panel and intermediate panels for securely sealing the rear panel against the intermediate panel.

5. The specimen bag according to claim 4, wherein the first and second adhesive seals extend between the right and left side weld lines.

6. The specimen bag according to claim 5, further including a release liner extending across and over each of the first and second adhesive seals, wherein the release liners prevent the first and second adhesive seals from prematurely closing the first and second chambers.

7. The specimen bag according to claim 6, further comprises a first set of score lines formed in and extending at least between the weld lines of the front panel and the intermediate panel.

8. The specimen bag according to claim 7, wherein the first set of score lines is formed between the lower terminal edges of the front and intermediate panels and the lower terminal edge of the rear panel.

9. The specimen bag according to claim 8, wherein the first set of score lines are in apposition to one another.

10. The specimen bag according to claim 9, further comprising a second set of score lines formed in and extending at least between the weld lines of the rear panel and the intermediate panel.

11. The specimen bag according to claim 10, wherein the second set of score lines is formed between the first and the second adhesive seals.

12. The specimen bag according to claim 11, wherein at least one of the first and second set of score lines is in the form of a grove defined by having a reduced thickness as compared to a thickness of the respective front and intermediate panel.

13. The specimen bag according to claim 3, wherein the first adhesive seal is applied to a surface of the intermediate panel in opposition to the front panel and at a location below the upper terminal edge of the front panel.

14. The specimen bag according to claim 4, wherein the second adhesive seal is applied to a surface of the intermediate panel in opposition to the rear panel and at a location below the upper terminal edges of the rear and intermediate panels.

15. The specimen bag according to claim 4, wherein the first and second adhesive seals is fabricated from a layer of pressure sensitive adhesive sealant material.

16. The specimen bag according to claim 6, wherein each release liner has a length which is less than the length of the adhesive seal which it covers.
